(19) United States
(12) Patent Application Publication
(10) Pub. No.: US 2008/0216593 A1
(43) Pub. Date: Sep. 11, 2008

(54) Device for promoting toe-off during gait

(76) Inventors: Stephen C. Jacobsen, Salt Lake City, UT (US); Fraser M. Smith, Salt Lake City, UT (US)

Correspondence Address: THORPE NORTH & WESTERN, LLP., P.O. Box 1219, SANDY, UT, 84091-1219, US

(21) Appl. No.: 12/072,127

(22) Filed: Feb. 22, 2008

Related U.S. Application Data
(60) Provisional application No. 60/902,994, filed on Feb. 22, 2007.

(51) Int. Cl. A61B 5/22

(52) U.S. Cl. 73/865.4

(57) ABSTRACT

Disclosed is a gait toe-off promoting device or a device configured to promote toe-off during gait comprising: (a) a first sensor located proximate the ball of a foot of an individual and configured to sense the force acting on the ball of the foot during a gait cycle and to provide a signal corresponding to the force; (b) a second sensor located proximate a pressure receiving surface of a toe of the foot of the individual and configured to measure the force acting on the toe of the foot during the gait cycle and to provide a signal corresponding to the force; (c) a control center configured to process the signals, as received from the first and second sensors, to obtain respective first and second measured values, wherein the control center is also configured to compare the first and second measured values to at least one pre-determined value programmed and stored within the control center; and (d) a feedback mechanism operably connected to and controlled by the control center and configured to notify the individual of improper toe-off during the gait cycle, wherein the feedback mechanism is activated as directed by the control center upon an unacceptable comparison of the measured values with the pre-determined value(s), thus making the individual aware of the need to correct future gait cycles.

### RELATED APPLICATIONS

This application claims the benefit of U.S. Provisional Application Ser. No. 60/902,994, filed Feb. 22, 2007, and entitled, “Device for Promoting Toe-Off During Gait,” which is incorporated by reference in its entirety herein.

### FILED OF THE INVENTION

The present invention relates generally to methods and devices for treating and preventing foot disorders, and more particularly to a method and device for promoting normal or exaggerated toe-off patterns during gait to treat and prevent bunions.

### BACKGROUND OF THE INVENTION AND RELATED ART

The term “bunion” is commonly used to describe the medical condition known as Hallux Valgus, which is one of the more widespread podiatric problems of our day. Hallux Valgus, or a bunion, is an enlargement of the joint of the forefoot, namely the metatarsophalangeal (MTP) joint located at the base of the big toe, that forms when the bone or tissue of this joint moves out of place. Although less common, bunions may also develop on the outside of the foot along the little toe. This type of bunion is referred to as a “bunionette.” Hereinafter, both bunions and bunionettes will be referred to generally as bunions.

Bunions form when the normal balance of forces that are exerted on the joints and tendons of the foot become disrupted. This can lead to instability in the joint and cause the deformity. The condition develops slowly and results from the gradual dislocation of the joint, usually because of instability during gait. In short, there is a displacement of the first metatarsal bone toward the mid-line of the body, and a simultaneous displacement of the big toe away from the mid-line (and toward the smaller toes). Bunions are often symptoms of faulty foot development and are usually caused by one or more of abnormal or improper gait, inherited foot type, and even the type, style, and fit of shoes worn. Wearing shoes that are too tight or cause the toes to be squeezed together explains the high prevalence of the disorder among women. Other causes of bunions are foot injuries, neuromuscular disorders, or congenital deformities. Those at higher risk of developing bunions are people who suffer from flat feet or low arches, arthritic patients, those with inflammatory joint disease, and those employed in occupations that place undue stress on the feet.

As an enlargement, and due to the displacement of bone and/or tissue, a bunion is a protuberance of bone that causes, or rather forces, the toe to bend toward the others. Stated another way, a bunion causes the toe to tilt away from the mid-line of the body, thus causing an often painful prominence of bone to appear at the joint site on the inside (medial) margin of the forefoot, which prominence of bone is manifested by the development of redness, swelling or contusion on the outside edge of the foot near the base of the big toe. Depending upon its size, this protuberance of bone at the MTP joint can significantly affect the biomechanical movement, or locomotion, of the person inflicted with the bunion. Indeed, walking, running, or even standing, all activities part of normal gait, at well as the participation in various activities can be extremely painful, especially since the metatarsophalangeal joint supports much of the body's weight during gait and various other movements. Moreover, the MTP joint itself may become stiff and sore, thus making even the wearing of shoes difficult or impossible.

Several methods and devices have been developed to relieve those suffering from bunions. Some of the more common approaches include, obtaining shoes of greater length and width to provide space for the bunion, applying a pad or other protective element to shield the bunion from the inside of the shoe, apply an ice pack to reduce swelling, padding and taping the bunion, taking medication, undergoing ultrasonic physical therapy, and/or wearing orthotic devices to help control the biomechanics of the foot. However, each of the treatment methods are merely a form of pain relief and do nothing to prevent bunions from initially forming.

If the above-described treatment methods are not enough to allow a person to perform normal everyday activities, such as if the bunions have progressed to the point of causing extreme pain and/or disabling the person, undergoing surgery to repair the MTP joint may be considered. As with any surgery, certain risks are involved that may or may not justify such action. Furthermore, surgery is something undertaken after the development of the bunion, and particularly the development of the bunion to an intolerable degree.

Based on the foregoing, there are several treatment options available to those having bunions. However, other than suggesting that a person wear certain shoes or an orthotic device, there are little known methods or devices for preventing bunions from developing altogether. It has been suggested that improper biomechanics can contribute to the development of bunions, thus one can deduce that preventing bunions is possible by ensuring continuous, proper biomechanical movements. In other words, the way a person walks, stands, and otherwise undergoes biomechanical locomotion can either contribute to, prolong, or prevent the development of bunions, depending upon the way the biomechanical locomotion is carried out.

The term “gait” is generally defined as the coordinated sequence of the various biomechanical movements of the lower limbs of a person undergoing locomotion. Gait is more typically described in terms of gait cycle due to the repetition of these movements during locomotion. For example, walking is a typical gait cycle and is used herein to describe the gait cycle.

Walking is divided into two phases. The first phase is the stance phase, which comprises the weight bearing portion of each gait cycle and is initiated by heel contact or heel-strike and ends with toe-off of the same foot. The second phase is the swing phase, which is initiated with toe-off and ends with heel-strike. Basically, the swing phase comprises the swinging of one limb to further locomotion while the contralateral limb remains grounded. The phrase “toe-off” refers to the instance of final contact between the toe and the floor. In normal gait, the point of final contact point between the toe and the floor generally occurs at the very front, bottom edge of the toe.

The stance phase comprises three segments, including (1) an initial double stance, (2) a single limb stance, and (3) a terminal double limb stance. The initial double stance segment accounts for approximately 10% of the gait cycle, as does the terminal double limb stance. The single limb stance accounts for a greater portion of the gait cycle, approximately 40%. As such, the stance phase accounts for a total of approximately 60% of the gait cycle, while the swing phase accounts for the remaining 40%.

The two limbs typically do not share the load equally during the double stance segments. Moreover, the load is typically fluctuating between limbs as gait progresses. During normal gait, ipsilateral swing temporally corresponds to single limb stance by the contralateral limb. If the velocity of gait is increased, variations begin to occur in the respective percentages of both the stance phase and the swing phase, and the duration of each aspect of the stance phase decreases until the walk becomes a run, in which case each of the double support periods are eliminated.

One gait cycle may be thought of in terms of a single stride. A stride may be defined as the distance between two successive placements of the same foot. Basically, a stride consists of two step lengths, left and right, each of which is the distance by which one foot moves forward in front of the other one. In normal gait, a person's step lengths are substantially similar to one another, whereas in pathological gait, or abnormal gait, it is possible for the two step lengths to differ.

More specifically, the gait cycle, or a single stride, comprises eight phases. The stance phase of the gait cycle comprises five sub-phases: (1) initial contact (the first 0-10% of the gait cycle), which occurs during initial double support and which includes initial contact, or heel-strike, and the loading response; (2) loading response (also within the first 0-10% of the gait cycle); (3) mid-stance (the next 10-30% of the gait cycle), which involves the progression of the body center of mass over the support foot and which trend continues through terminal stance; (4) terminal stance (the next 30-50% of the gait cycle), which begins with heel rise of the support foot and terminates with contralateral foot contact; and (5) pre-swing (the next 50-60% of the gait cycle), which begins with terminal double support and ends with toe-off of the ipsilateral limb.

The swing phase of the gait cycle comprises the remaining three sub-phases: (1) initial swing (the next 60-73% of the gait cycle); (2) mid swing (the next 73-87% of the gait cycle); and (3) terminal swing (the remaining 87-100% of the gate cycle), each of which collectively effect foot clearance and advancement of the trailing limb.

The development of bunions may be a result of improper biomechanics during gait, particularly during the pre-swing phase where toe-off is involved, thus leading to pathological gait. Pathological gait describes altered gait patterns. Normally, the big toe will bend at least 65 degrees during toe-off. However, with the presence of a bunion, the big toe cannot function properly. This typically results in the person altering his or her biomechanics or gait to compensate for the bunion by walking in an exaggerated manner with the big toe, or rather the entire foot, turned out so that the big toe does not have to bend as far during gait. Gait may also be altered by transferring the weight of the body to the bunion or the ball of the foot to remove pressure on the toe altogether. Unfortunately however, altering the gait in such ways adversely affects the person by steadily forcing the big toe even farther out, and thus worsening the condition. Therefore, improper toe-off can not only contribute to the development of bunions, but it can also worsen the condition of existing bunions and contribute to pathological gait as the normal balance of forces that are exerted on the joints and tendons of the foot are disrupted, thus causing instability in the MTP joint, as stated above. Conversely, proper toe-off may prevent the development of bunions and/or reduce the pain and suffering associated with existing bunions, as well as to promote normal and proper gait.

### SUMMARY OF THE INVENTION

In light of the problems discussed above and the apparent deficiencies inherent in the prior art, the present invention seeks to overcome these by providing a method and device for encouraging or promoting therapeutic gate, and particularly normal or exaggerated toe-off during the gait of an individual for the purpose of preventing the formation of bunions and for slowing or halting the progress of existing bunions. The present invention device also functions to discourage seemingly desirous and self-interested pathological gait or the alteration of normal gait patterns undertaken by an individual in response to one or more existing bunions to lessen the pain and influence of the bunion during gait. Discouraging pathological gait, despite the individual's desire to do otherwise, helps to reduce the likelihood that the condition of the bunion(s) will worsen.

In accordance with the invention as embodied and broadly described herein, the present invention features a gait toe-off promoting device or a device configured to promote toe-off during gait comprising: (a) a first sensor located proximate the ball of a foot of an individual and configured to sense the force acting on the ball of the foot during a gait cycle and to provide a signal corresponding to the force; (b) a second sensor located proximate a pressure receiving surface of a toe of the foot of the individual and configured to measure the force acting on the toe of the foot during the gait cycle and to provide a signal corresponding to the force; (c) a control center configured to process the signals, as received from the first and second sensors, to obtain respective first and second measured values, wherein the control center is also configured to compare the first and second measured values to at least one pre-determined value programmed and stored within the control center; and (d) a feedback mechanism operably connected to and controlled by the control center and configured to notify the individual of improper toe-off during the gait cycle, wherein the feedback mechanism is activated as directed by the control center upon an unacceptable comparison of the measured values with the pre-determined value(s), thus making the individual aware of the need to correct future gait cycles.

The present invention further features a device configured to promote toe-off during gait comprising: (a) a first sensor located proximate a pressure receiving surface of the ball of a foot of an individual and configured to sense the force acting on the ball of the foot during a gait cycle and to provide a signal corresponding to the force; (b) a second sensor located proximate a pressure receiving surface of a toe of the foot of the individual and configured to measure the force acting on the toe during the gait cycle, as well as to provide a signal corresponding to the force; (c) a control center configured to process the signals, as received from the first and second sensors, to obtain respective first and second measured values, wherein the control center is also configured to compare the first and second measured values and to activate a feedback device depending upon the results of the comparison; and (d) an interface medium configured to house and support at least the first and second sensors.

In one exemplary embodiment, the device is self-contained and configured for placement or location between the ball of the foot and the toes about the metatarsophalangeal (MTP) joint, with part of the device comprising a pressure or force sensor that is juxtaposed to the ball of the foot, and with another part of the device comprising a similar pressure or force sensor being juxtaposed to the bottom of one or more of the toes, particularly the big toe. The sensors are electrically coupled to a control center that receives and interprets or processes the signals from the sensors and that controls the activation of a feedback mechanism in the event of an unacceptable pressure differential. The feedback mechanism may be activated immediately upon a current pressure differential or it may be activated in a delayed manner, such as after several pressure differentials have been processed whose average comprises an unacceptable pressure differential.

In another exemplary embodiment, the device comprises different modules that are operably coupled together, such as a shoe that comprises sensors in its sole that are adjacent the ball of the foot and the toes when the shoe is placed on the foot. Other exemplary devices are also contemplated that are configured to locate suitable sensors about the ball of the foot and the toes.

The present invention further features a method for promoting toe-off during gait comprising: (a) positioning a first sensor proximate the ball of a foot of an individual, wherein the first sensor is configured to sense the force acting on the ball of the foot during a gait cycle and to provide a signal corresponding to the force; (b) positioning a second sensor proximate the toe of the foot, wherein the second sensor is configured to sense the force acting on the toe during the gait cycle and to provide a signal corresponding to the force; (c) transmitting the signals to a control center for processing, wherein the control center assigns a measured value to the signals; (d) comparing the measured values to at least one pre-determined value; and (e) activating a feedback mechanism in the event the recited step of comparing results in at least one of the measured values being unacceptable.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 illustrates a block diagram of a normal and/or therapeutic gait toe-off promoting device according to a first exemplary operating configuration, wherein the various components of the gait toe-off promoting device are all contained in a single structural member;

FIG. 2 illustrates a block diagram of a gait toe-off promoting device according to a second exemplary operating configuration, wherein the first and second sensors and the feedback mechanism are all located or contained within a first structural member, namely the interface medium, while the control center and its components, namely the processor, the transceiver, the memory storage device, and the power source, are all contained in a separate structural member;

FIG. 3 illustrates a graph of the forces acting on the ball of the foot and the toe over time during a gait cycle;

FIG. 4 illustrates an exploded top view of a gait toe-off promoting device according to a first exemplary embodiment, wherein the gait toe-off promoting device employs the operating configuration depicted in FIG. 1;

FIG. 5 illustrates an exploded perspective view of the gait toe-off promoting device of FIG. 4 as interfacing with a foot;

FIG. 6 illustrates a top view of a gait toe-off promoting device according to a second exemplary embodiment, wherein the gait toe-off promoting device employs the operating configuration depicted in FIG. 1 and is contained within a shoe insert;

FIG. 7 illustrates an exploded perspective view of the gait toe-off promoting device of FIG. 6 as interfacing with a foot; and

FIG. 8 illustrates various sub-phases of a gait cycle and the associated forces acting on the foot during each sub-phase.

### DETAILED DESCRIPTION OF EXEMPLARY EMBODIMENTS

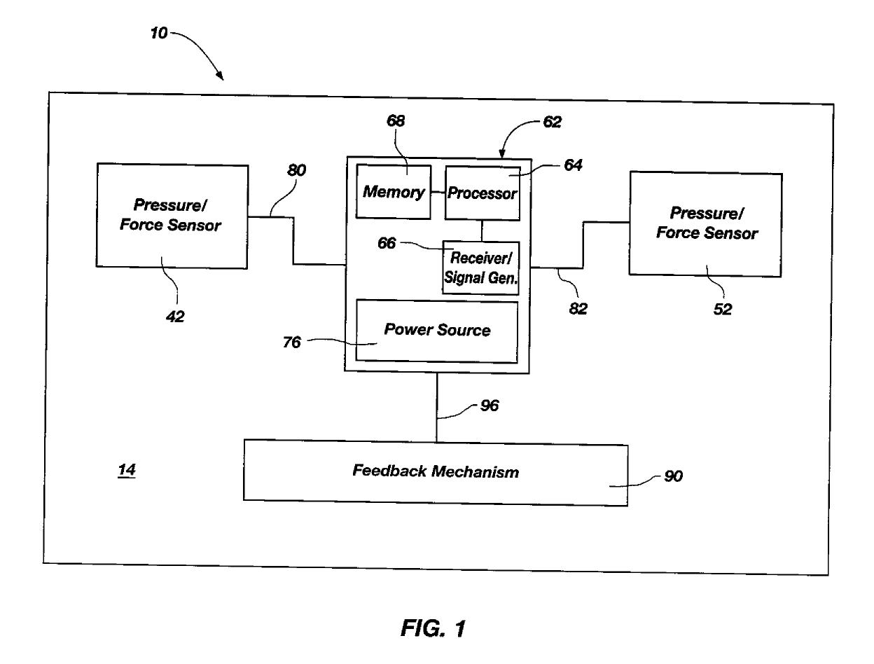

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art practice the invention, it should be understood that other embodiments may be realized and that various changes to the invention may be made without departing from the spirit and scope of the present invention. Thus, the following more detailed description of the embodiments of the present invention, as represented in FIGS. 1 through 8, is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

The present invention features a method and device or system for promoting and/or encouraging normal or therapeutic gait patterns in humans, and thus discouraging seemingly desirable, yet injurious pathological gait or the alteration of normal gait patterns for the purpose of lessening the pain experienced during normal gait as a result of the presence of one or more bunions. More specifically, the present invention features a device configured to promote normal and/or therapeutic biomechanical patterns during gait by encouraging proper or normal toe-off, or even exaggerated toe-off, preferably with each gait cycle. The term “gait” is used herein to describe one form of locomotion that the present invention may be adapted to monitor, namely walking. The present invention is also adaptable and intended for use with other forms of locomotion, as will be recognized by one skilled in the art, although each form is not specifically recited herein. Examples of other forms of locomotion may include jogging, sprinting, climbing, etc.

In general, the present invention normal and/or therapeutic gait toe-off promoting device comprises pressure/force sensors having the ability to monitor the pressure/force applied to the ball of the foot and also the bottom of the toes during the gait cycles of the individual wearing the device. Each sensor sends out one or more signals corresponding to the forces applied to the sensors. Utilizing a control center, the signals from the sensors are received and processed to determine a measurable corresponding force or pressure value. The control center further processes the signals to determine the pressure/force differential, if any, existing between the big toe and the ball of the foot during a gait cycle, as indicated by the difference in the signal values. A certain pressure differential will indicate when the individual is not conducting proper toe-off. This measured pressure differential is then compared to one or more pre-determined or programmed range of acceptable or unacceptable pressure differentials stored by the control center to determine whether the pressure differential is sufficient to notify the individual of his or her exercise of improper biomechanics, and particularly improper toe-off, during gait. If the pressure differential falls within an unacceptable range, the device is configured to notify the individual of his or her improper biomechanics using electrical or mechanical means, as provided by a feedback mechanism, thus encouraging the individual to alter his or her gait patterns, and particularly his or her toe-off patterns, to be more normal or even exaggerated. In essence, if, during a current gait cycle or over a pre-determined number of gait cycles, the person is not toeing-off properly, an unacceptable pressure differential will exist between the ball of the foot and the bottom of the toes that will trigger the control center to activate the feedback mechanism of the device, thus notifying the individual of the unacceptable pressure differential and his or her improper toe-off patterns. As indicated above, the practice of harmful abnormal gait patterns for one or more reasons is known as pathological gait. As such, the present invention device functions to encourage the individual to toe-off properly and to exercise normal and proper biomechanics during gait, as well as to discourage self-interested pathological gait. The present invention normal and/or therapeutic gait toe-off promoting device may also be configured to not only encourage the individual to toe-off, but to exaggerate toe-off. Each of these concepts and the particulars of the various embodiments of the normal gait promoting devices are explained in greater detail below.

With reference to FIG. 1, illustrated is a block diagram of a normal and/or therapeutic gait toe-off promoting device (hereinafter “gait toe-off promoting device”) according to a first exemplary operating configuration, wherein the various components of the gait toe-off promoting device are all contained in a single structural member. Specifically, FIG. 1 illustrates the gait toe-off promoting device 10 as comprising an interface medium 14 that functions as the structural member designed to house and/or support the various components of the gait toe-off promoting device 10, namely the first and second sensors, 42 and 52, respectively, the control center 62, and the feedback mechanism 90. The interface medium 14 is configured to couple to or otherwise interface with the foot of an individual with the first and second sensors 42 and 52 and the feedback mechanism 90 properly positioned for the purposes described herein.

As supported within or about the interface medium 14, the first sensor 42 is configured to be placed on or about the ball of the foot of the individual in a position aft of the MTP joint. Particularly, the first sensor 42 is located proximate the lower surface of the ball of the foot, or rather the pressure receiving surface of the ball of the foot. Once positioned, the first sensor 42 functions or is designed and configured to sense the various pressures or forces acting on the ball of the foot during gait, which forces are applied through the interaction of the ball of the foot with the ground or other surface, and to supply or transmit a signal corresponding to the applied force. During normal gait, the greatest magnitudes of pressure or force that are applied to the ball of the foot occur during the mid-stance and terminal stance sub-phases of the gait stance phase, with the forces being the greatest during the terminal stance sub-phase. As normal gait progresses to the pre-swing sub-phase, where toe-off occurs, the forces on the ball of the foot drop off until eventually there are zero forces acting on the ball of the foot, which marks the beginning of the swing phase of the gait cycle.

Also as supported within or about the interface medium 14, the second sensor 52 is configured to be placed on or about the big toe of the foot of the individual in a position forward the MTP joint. Particularly, the second sensor 52 is located proximate the lower surface of the big toe, or rather the pressure receiving surface of the big toe. Once positioned, the second sensor 52 functions or is designed and configured to sense the various pressures or forces acting on the bottom of the big toe during gait, which forces are applied through the interaction of the big toe with the ground or other surface, and to supply or transmit a signal corresponding to the applied force. During normal gait, the greatest magnitudes of pressure or force that are applied to the big toe of the individual occur during the terminal stance and pre-swing sub-phases of the gait stance phase, with the forces being the greatest during the pre-swing sub-phase and then dropping off and zeroing during the swing phase of the gait cycle.

During pathological gait, or in those circumstances where an individual alters his or her normal gait patterns for a self-interested reason, such as to minimize the pain experienced during gait due to the existence of a bunion, the individual may purposely reduce the forces allowed to act on the big toe so that they are nominal. In extreme cases, the individual may eliminate the forces acting on the big toe altogether, depending upon the condition and severity of the bunion. Action to this end effectively reduces the benefit of or eliminates altogether the pre-swing sub-phase of the gait cycle, thus negating any advantages that the pre-swing sub-phase and proper toe-off may have for the individual. Rather than carrying out the full pre-swing sub-phase, most or all of the forces acting on the foot during the latter sub-phases of the stance phase leading up to and in preparation for the swing phase will be concentrated on the balls of the feet. For example, an individual with bunions may try to forego toeing-off and walk mostly or entirely on the balls of his or her feet, thus favoring the big toe and allowing it to bear only a nominal portion of the individual's body weight or no weight at all. Or, a person may try to walk with relaxed toes, thus not properly flexing the big toe during the pre-swing sub-phase and allowing the MTP joint to adversely flex. By walking mostly or entirely on the balls of the feet, or by relaxing the toes so as to induce improper toe flexure, the individual is effectively altering normal gait patterns, or in other words exercising a form of pathological gait, by not exercising proper or use of the toe(s). More specifically, the individual is failing to properly toe-off during the pre-swing sub-phase of gait. Improper toe off, or the elimination of toe-off altogether, although possibly providing immediate relief of the effects of the bunion, may cause the condition of the bunion to worsen, often unbeknownst to the individual. This is especially true if the gait cycle is repeated in such manner for an extended period of time. In altered or pathological gait patterns such as these, the second sensor 52 may sense and transmit a signal corresponding to a nominal force or no force at all, depending upon the severity or extent of the gait alterations, in which case the first sensor 42 on the ball of the foot may sense and transmit a signal corresponding to a measurable force through to the swing phase of the gait cycle.

The gait cycle and associated forces described above are repeated for the contralateral foot.

Correlating with their sensing function, the first and second sensors 42 and 52 are also configured to transmit respective signals to the control center 62, which signals correspond to the sensed pressure or force acting on the sensors through impact of the foot with the ground or other surface. The first sensor 42 is electrically and operably coupled to the appropriate components of the control center 62 through interconnect 80. Likewise, the second sensor 52 is electrically and operably coupled to the appropriate components of the control center 62 through interconnect 82. Interconnects may be any wire or other similar means capable of carrying the signal from the sensors 42 and 52 to the necessary components of the control center 62. First and second sensors 42 and 52 may also be wireless sensors, thus eliminating the need for a physical connection to the control center 62.

It is noted that the present invention is not limited to the use of any particular type of sensor. Indeed, the first and second sensors 42 and 52 may comprise any type of pressure or force or impact-sensitive sensors commonly available in the art capable of sensing the pressures or forces acting on the foot of an individual during gait or other locomotion. In addition, it is intended that the first and second sensors 42 and 52 be sized and configured accordingly for placement proximate the ball of the foot and the big toe, respectively, of the individual. In one exemplary embodiment, the first and second pressure sensors 42 and 52 are comprised of piezoelectric sensors, as commonly known. In short, the piezoelectric sensors act as transducers that turn the applied forces experienced during gait, or in other words mechanical stress, into an electrical charge, which is then turned into a voltage. The resulting voltage is a function of the applied force. This voltage is then transmitted to the control center 62 where it is further processed. In another exemplary embodiment, the first and second sensors 42 and 52 may comprise foot force sensors, also as commonly known in the art. Other types of sensors that are capable of sensing or measuring force on the foot of an individual may also be implemented and practiced with the present invention, as will be apparent to those skilled in the art, such as various contact switches. As such, the first and second sensors 42 and 52 are only generically described herein.

The control center 62 comprises all of the necessary components, such as receivers, transmitters, logic circuitry, processors, storage devices, filters, and/or signal generators, etc., to operate the gait toe-off promoting device 10, as commonly known in the art. In the embodiment shown in FIG. 1, the control center 62 comprises a receiver/signal generator or transceiver 66 configured to receive the signals sent from the first and second sensors 42 and 52. The transceiver 66 may be any known in the art capable of operating with the selected type of sensor.

As the signals are received from the first and second sensors 42 and 52, they are further processed by the control center 62 by a processor 64. The processor 64 may also be any type of processor capable of analyzing and comparing the received signals, as well as performing other related functions, such as triggering the activation of the feedback mechanism 90. The control center 62 further comprises a memory storage device 68 that stores one or more pre-determined pressure or force values. These values may be programmed into the control center 62 for later comparison with currently measured values of the various sensors.

The control center 62 may also be configured or calibrated differently in order to allow different individuals to use the device 10. Obviously, individuals vary in their size, their weight, in their individual gait patterns, etc. As such, the device 10 is intended to be programmable to accommodate different users. With each user, the device may be calibrated so that the feedback mechanism 90 is activated within a reasonable range of parameters. However, the parameters of the device 10 set for one person may be inadequate or inappropriate for another. Therefore, it is advantageous to provide a control center 62 that may be calibrated and recalibrated as needed.

As indicated, the control center 62 receives the signals from each of the first and second sensors 42 and 52, corresponding to the force or pressure acting on the ball of the foot and the big toe, respectively. Once received, the signals from the first and second sensors 42 and 52 are analyzed, measured, and assigned a value. These values are then processed and analyzed in one or more ways to determine whether the feedback mechanism 90 should be activated to notify the individual of improper toe-off.

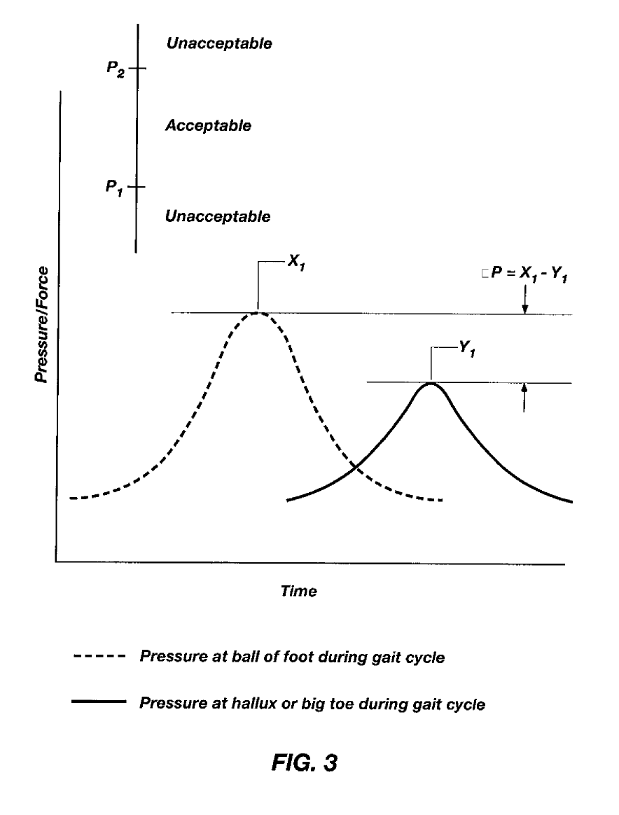

In one exemplary embodiment, and with reference to FIGS. 1 and 3, the values from the sensors 42 and 52 are compared against one another to determine whether a pressure or force differential exists between the force(s) acting on the ball of the foot to those acting on the big toe of the foot as represented by the measured values. If a pressure differential exists, which will typically be the case in both normal and pathological gait, the control center 62, via the processor 66, will determine the degree of difference between the two measured values and assign this difference a value that may be referred to as a measured pressure differential value, and identified as ΔP, measured in terms of absolute value. Once a measured pressure differential value ΔP is determined, the control center 62 is configured to compare this value to one or more, and preferably a range, of pre-determined pressure differential values stored within the memory storage device 68 of the control center 62. The pre-determined pressure differential values may be in the form of acceptable values, unacceptable values, or a combination of these. If in the form of acceptable values, the feedback mechanism 90 will be triggered if the measured pressure differential value ΔP does not match one of the pre-determined values. Similarly, if in the form of unacceptable values, the feedback mechanism 90 will be triggered if the measured pressure differential value ΔP matches one of the pre-determined values. If a combination of acceptable and unacceptable values, the feedback mechanism 90 will be triggered depending upon whether the measured pressure differential value ΔP registers in the acceptable or unacceptable range. For example, if in FIG. 3, x₁ represents the peak force value received from the first sensor 42 (which value is representative of the peak pressure or force acting on the ball of the foot during the gait cycle), and if y₁ represents the peak force value received from the second sensor 52 (which value is representative of the peak force or pressure acting on the big toe during the gait cycle), and also if ΔP=|x₁−y₁| (absolute value) represents the measured pressure differential value, and if P₁=0, and if P₂ represents the upper limit of an acceptable range, the feedback mechanism 90 will be triggered if ΔP>P₂. Stated another way, the feedback mechanism will not be triggered if ΔP<P₂.

Alternatively, if a pressure differential exists, the control center 62 may be configured to separately compare the measured pressure or force values obtained from the signals provided by the first and second sensors 42 and 52 with separate sets of pre-determined pressure or force values correlating to the ball of the foot and the big toe. Thus, the control center 62 may be configured to compare the measured pressure or force value obtained from the first sensor 42 with a first set of pre-determined pressure or force values stored within the memory storage device 68 of the control center 62. Likewise, the control center 62 may be configured to compare the measured pressure or value obtained from the second sensor 52 with a second set of predetermined pressure or force values, also stored within the memory storage device 68. To trigger the feedback mechanism 90, the control center 62 determines whether the two separately compared values fall within acceptable or unacceptable ranges. This is strictly a direct comparison of the measured values to the programmed stored values.

In still another alternative embodiment, similar to the one just discussed, the feedback mechanism 90 may be triggered if the control center 62 determines that the two separately compared values fall within acceptable or unacceptable ranges as based on the reading of the other. For example, if xi represents the value received from the first sensor 42 (which value is representative of the pressure or force acting on the ball of the foot), and if y₁ represents the value received from the second sensor 52 (which value is representative of the force or pressure acting on the big toe), and P represents an acceptable variation that y₁ may be from x₁, the feedback mechanism 90 will not be triggered if y₁=x₁±P. Stated another way, the feedback mechanism 90 may be triggered if y₁<x₁−P; or if y₁>x₁+P. It is noted herein that the control center 62 may be configured to activate the feedback mechanism 90 only in the event y₁<x₁−P, since this would signal that the pressure acting on the toe is substantially less than the pressure acting on the ball of the foot, a condition indicative of improper toe-off. The value of P may be pre-determined and programmed into the control center 62.

In still another alternative embodiment, the processor 64 may simply determine if the pressure one the big toe is below a certain threshold value, and trigger the feedback mechanism 90 accordingly.

Preferably, the processor 66 will analyze the peak pressure applied to both the ball of the foot and the big toe during a given number of gait cycles. Indeed, as an individual initiates locomotion and proceeds through the various phases of the gait cycle, the ball of the foot and the big toe will experience different magnitudes of pressure, even in normal gait. As such, the first and second sensors 42 and 52 may transmit a number of different signals corresponding to the number of different pressure or force magnitudes sensed. The processor 66 should be configured to analyze or sort through these different signals to determine the peak or high force magnitude applied to the respective areas of the foot and use these peak magnitudes for comparison purposes. Alternatively, the processor 66 may take several pressure readings by the first and second sensors 42 and 52 that occur over a single gait cycle (e.g., one stride) or a number of gait cycles (e.g., multiple strides), and average these to determine the respective measured values from each sensor to be compared. This average value may be used in a similar manner as the peak value discussed above, namely compared to the pre-determined, stored value.

Powering the gait toe-off promoting device 10 is power source 76. Power source 76 may be any known in the art. In one exemplary and preferred embodiment, the power source 76 comprises a battery-type power source. Other types of power sources known in the art may be used and are contemplated herein.

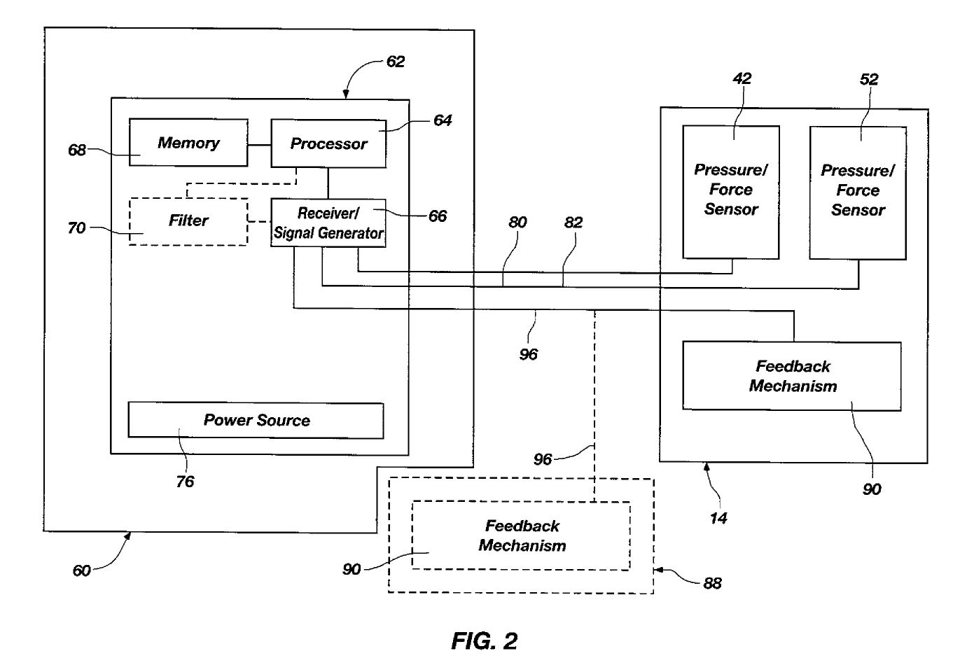

The device 10 further comprises, and the interface medium 14 further supports, feedback mechanism 90 that is configured to notify the individual of improper or unacceptable gait patterns, and particularly improper or unacceptable toe-off during gait. Feedback mechanism 90 is controlled and activated by the control center 62 and is coupled thereto via interconnect 96. The control center 62 is configured to trigger or activate the feedback mechanism 90 in the event the measured pressure or force values from the first and second sensors 42 and 52 fall without acceptable ranges in one or more ways, as discussed above. In one exemplary embodiment, the feedback mechanism 90 comprises a vibrating device that vibrates at a suitable amplitude and frequency to notify the user of improper toe-off. In another exemplary embodiment, the feedback mechanism 90 may comprise an audible device capable of producing an audible sound that warns the individual of improper toe-off. Other types of feedback mechanisms may be utilized that are not discussed herein, but that will be apparent to those skilled in the art. The feedback mechanism 90 may be contained within the interface medium 14, or it may be a separate stand-alone structure, as shown in FIG. 2. In addition, the feedback mechanism 90 may be powered by the power source 76 of the control center 62, or by its own power source.

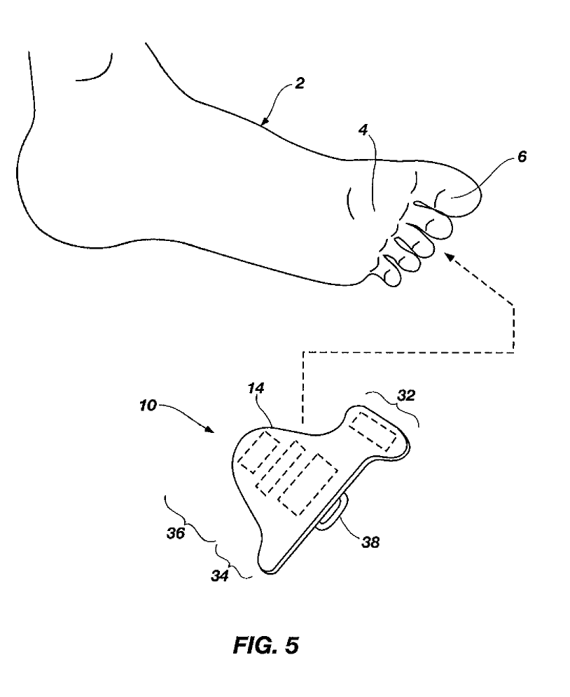

The interface medium 14 comprises any suitable structural member capable of housing and supporting some or all of the components of the device. In one aspect, the interface medium 14 comprises a structure configured to house all of the components of the device, such as that shown in FIGS. 3 and 5 and discussed below. In another aspect, the interface medium 14 comprises a structural member configured to house and support only the sensors 42 and 52 and the feedback mechanism 90 in a location about the MTP joint. In this configuration, the control center 62, with its associated power source, are electrically coupled to the sensors 42 and 52 and the feedback mechanism 90, but are not contained in the interface medium 14 (see FIG. 2).

The interface medium 14 may be formed of any suitable material. In one exemplary and preferred embodiment, the interface medium 14 is comprised of an elastomer material configured to flex and bend with the movements of the foot. In another embodiment, the interface medium 14 may be comprised of any material used to form a shoe sole or a shoe insert.

With reference to FIG. 2, illustrated is a block diagram of a gait toe-off promoting device 10 according to a second exemplary operating configuration, wherein the first and second sensors 42 and 52 and the feedback mechanism 90 are all located or contained within the interface medium 14, while the control center 62 and its components, namely the processor 62, the transceiver 66, the memory storage device 68, and the power source 76, are all contained in a separate structural member, labeled as member 60. The first and second sensors 42 and 52 and the feedback mechanism 90 are electrically and operably coupled to the control center 62 through a series of interconnects, namely interconnects 80, 82, and 96, respectively. Again, the interface medium 14 is configured for placement about the foot of the user such that the first sensor 42 is proximate the ball of the foot and the second sensor 52 is proximate the big toe of the foot. However, the member 60, as a separate structure, may be located in a location distal from the interface medium 14, such as on the top of a shoe, etc.

FIG. 2 further illustrates that the feedback mechanism 90 may also be contained in its own structure, shown as member 88. In this configuration, the feedback mechanism 90 is separate from the interface medium 14 and the member 60 housing the control center 62. It is noted herein that FIGS. 1 and 2 simply illustrate some of the exemplary operating configurations that the device 10 may be found in. Others not specifically recited herein will be apparent and obvious to those skilled in the art and are therefore contemplated herein. It is also noted herein, that FIGS. 1 and 2 are general representations of the basic components that will typically be included in the gait toe-off promoting device as contemplated herein. Other components may be added or some deleted or modified in one or more ways to achieve a different layout for the gait toe-off promoting device, such as the type of circuitry employed, the type and number of sensors used, the type of feedback mechanism employed, the type of power source, etc., each of which will be recognized by one skilled in the art.

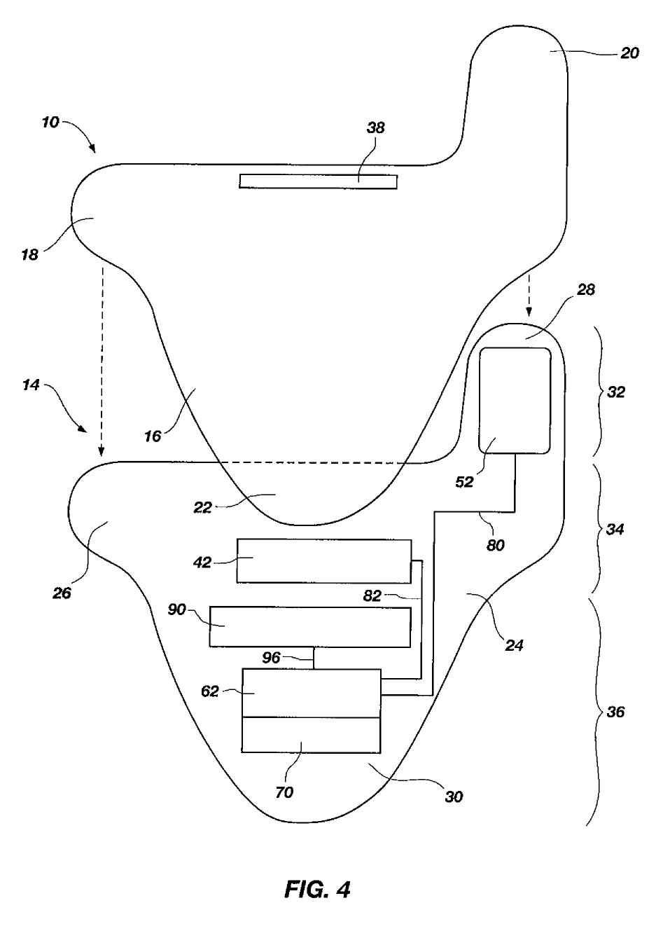

With reference to FIG. 4, illustrated is an exploded top view of a gait toe-off promoting device according to a first exemplary embodiment, wherein the gait toe-off promoting device employs the operating configuration depicted in FIG. 1, particularly wherein the various components of the gait toe-off promoting device are all contained in a single structural member. Specifically, FIG. 4 illustrates the gait promoting device 10 as comprising an interface medium 14 having an upper support member 16 configured to be coupled to a lower support member 24, thus forming an interior portion for housing and supporting the various components of the device 10. The upper support member 16 comprises a ball portion 18 for positioning about the ball of the foot, a toe extension portion 20 for positioning about the big toe of the foot, and a control center portion region 22 for housing the control center 62 and its components. Likewise, the lower support member 24 comprises a ball portion 26 for positioning about the ball of the foot, a toe extension portion 28 for positioning about the big toe of the foot, and a control center portion 30 for housing the control center 62 and its components, each of which correspond to the like portions of the upper support member 18. In effect, the upper support member 16 fits over the lower support member 24 and is secured thereto to support the components of the device 10 in their proper place. Specifically, the first sensor 42 is contained within the ball region 34, the second sensor 52 is contained within the toe region 32, and the control center 62 is contained within the control region 36 of the interface medium 14. Interconnects 80, 82, and 96 span between these regions to connect the various components. Although the feedback mechanism 90 is shown located within the control region 36, this is not limiting in any way. Indeed, since the feedback mechanism 90 functions simply to alert the user of improper toe-off, it may be located anywhere within the interface medium 14.

With reference to FIGS. 4 and 5, as assembled, the device 10, and particularly the interface medium 14, may interface with a foot 2 of the user. As the interface medium 14 interfaces with the foot 2, particularly about the MTP joint 8, the first and second sensors 42 and 52 will be in their proper places to sense the pressures or forces acting on the ball 4 of the foot and the big toe 6 of the foot, respectively. The interface medium 14 may couple to the foot using any known means in the art. In one exemplary embodiment, the interface medium further comprises a band element 38 coupled to the outer surface of the interface medium 14. The band element 38 is configured to engage one or more toes of the user, thus securing the interface medium 14 to the foot 2 in another exemplary embodiment, the interface medium 14 comprises an adhesive layer that allows the interface medium 14 to be removably coupled to the foot 2. Other types of securing devices or systems may be used that are not specifically recited herein.

The interface medium 14, as shown in FIGS. 4 and 5, is designed to be worn by an individual with or without shoes. As the individual walks, the interface medium 14 is designed to flex and bend so that the first and second sensors 42 and 52 contained therein remain in place. It is noted that the particular shape and configuration of the device 10 illustrated in FIGS. 4 and 5 is illustrative only and is not meant to be limiting in any way. There are several different structural shapes and component configurations that may be employed to practice the present invention and each of these are intended to fall under the scope of the claims as presented herein.

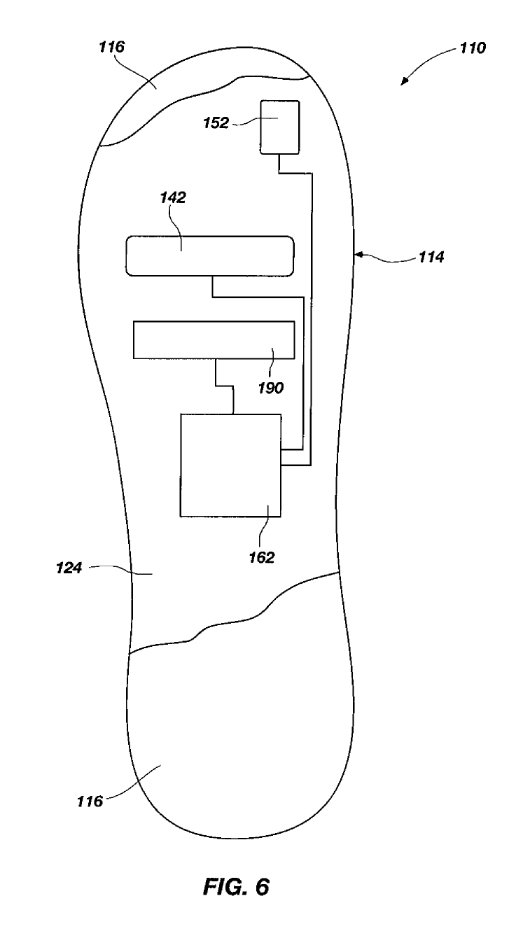

FIG. 6 illustrates a top view of a gait toe-off promoting device according to a second exemplary embodiment, wherein the gait toe-off promoting device again employs the operating configuration depicted in FIG. 1, particularly wherein the various components of the gait toe-off promoting device are all contained in a single structural member. Specifically, FIG. 6 illustrates the gait promoting device 110 as comprising an interface medium 114 in the form of a shoe insert configured to be inserted into the bottom of a shoe as commonly known. The interface medium 14, or shoe insert, has an upper support member 116 configured to be coupled to a lower support member 124, thus forming an interior portion for housing and supporting the various components of the device 110, namely the first and second sensors 142 and 152, the control center 162, and the feedback mechanism 190, similar to the embodiment described in FIGS. 4 and 5.

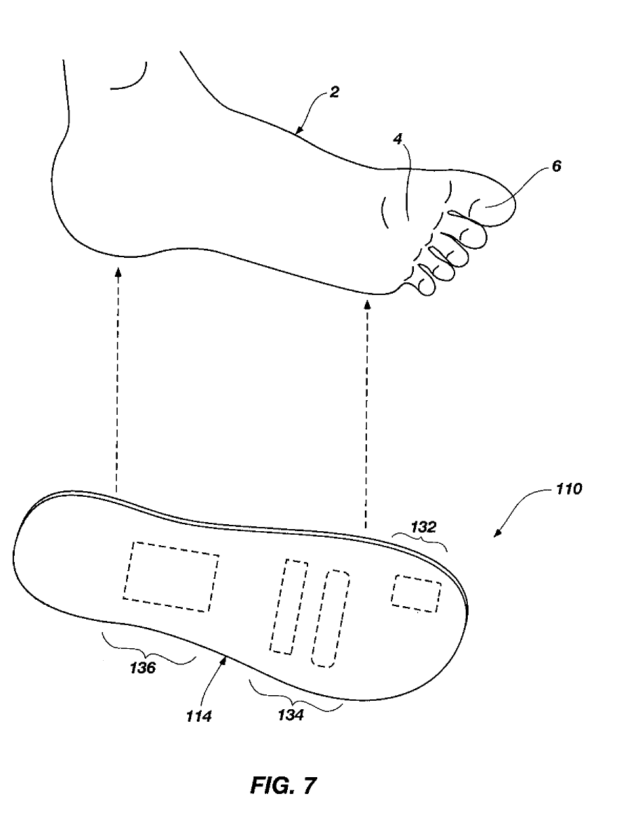

With reference to FIGS. 6 and 7, as assembled, the device 10, and particularly the interface medium 114, may interface with a foot 2 of the user by being inserted into a suitable shoe (shoe not shown). As the individual places the shoe on his or her foot, the interface medium 14 interfaces with the foot 2, particularly about the MTP joint 8, thus positioning the first and second sensors 142 and 152 proximate the ball 4 and big toe 6 of the foot 2, respectively, in order to sense the pressures or forces acting on the ball 4 and big toe 6 of the foot 2. As can be seen, the first sensor 142 is located within the toe region 132 of the interface medium 114, the second sensor 152 is located within the ball region 134 of the interface medium 114, and the control center 162 is located within the control region 136 of the interface medium 114. Also, in this embodiment, the feedback mechanism 190 is located within the ball region 134.

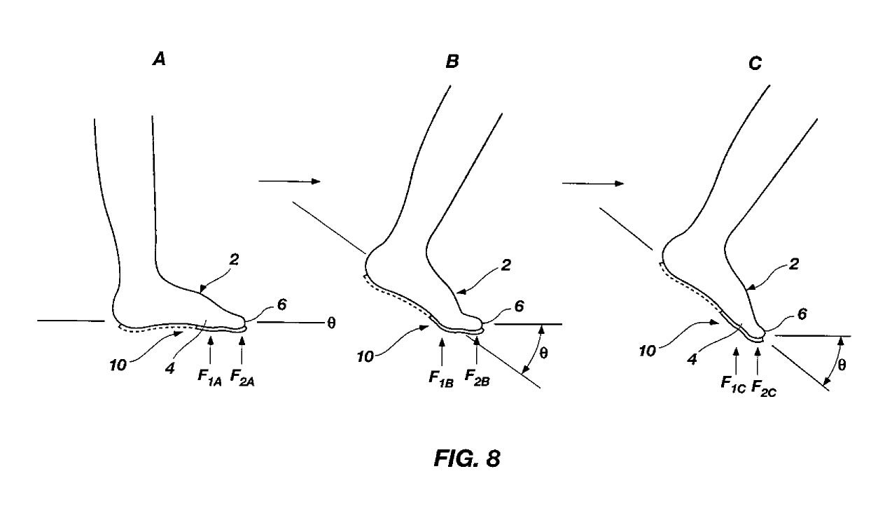

With reference to FIG. 8, illustrated are several different views depicting the present invention gait toe-off promoting device being worn by an individual undergoing the latter sub-phases of the stance phase of a normal gait cycle. In FIG. 8-A, the individual is shown in the mid-stance sub-phase where the ball 4 of the foot 2 is subjected to a force F_{1A}, and where the toe 6 is subject to a force F_{2A}. Force F_{1A} and F_{2A} are applied to the device 10, thus effectively operating the device. In this sub-phase, the longitudinal axis of the foot 2 is parallel or substantially parallel to the ground (θ≈0).

FIG. 8-B illustrates the individual in the terminal stance sub-phase where the ball 4 of the foot 2 is subject to the greatest force, shown as force F_{1B}, and wherein the toe 6 is also subject to an increased force, shown as force F_{2B}, comparative to the force F_{2A} applied to the toe 6 in the mid-stance sub-phase. Again, force F_{1B} and F_{2B} are applied to the device 10, thus effectively operating the device. As can be seen, in the terminal stance sub-phase, the angle θ between the longitudinal axis of the foot 2 and the ground is significant. However, the longitudinal axis of the toe remains parallel or substantially parallel with the ground.

FIG. 8-C illustrates the individual in the pre-swing sub-phase where the ball 4 of the foot 2 has a zero force, shown as force F_{1C}, and wherein the toe 6 is subject to the greatest force, shown as force F_{2C}. Again, force F_{1C} and F_{2C} are applied to the device 10, thus effectively operating the device. In this sub-phase, the angle θ between the longitudinal axis of the foot 2 and the ground is the greatest of the gait cycle. Moreover, the longitudinal axis of the toe 6 is shown parallel or substantially parallel with the longitudinal axis of the foot 2, thus indicting proper toe-off.

Improper toe-off occurs when the individual diminishes or eliminates the presence of the pre-swing sub-phase (FIG. 8-C). During abnormal or pathological gait, an individual may propel himself or herself into the swing phase (not shown) by diminishing the pre-swing phase, thus conducting partial toe-off. A worse scenario is when the individual propels himself or herself into the swing phase directly from the terminal stance sub-phase (FIG. 8-B), thus bypassing the pre-swing phase, and toe-off, altogether. Even though providing for temporary relief during gait, either of these scenarios can significantly contribute the development of bunions, or worsen the condition of an existing bunion. Therefore, the present invention gait toe-off promoting device functions to discourage such scenarios and to promote a more normal gait pattern, which if practiced, can reduce the painful effects of bunions.

The present invention provides several advantages over prior related devices and systems. First, the present invention device promotes proper biomechanics during gait or other locomotion by notifying the individual when improper biomechanics are being performed so that appropriate correction before the next gait cycle can be made. Second, the present invention device emphasizes the importance proper gait patterns, and namely the transfer of weight from the ball of the foot to the toes of the foot, and particularly the big toe, to achieve proper toe-off. Emphasizing proper or even exaggerated toe-off can significantly reduce the effects of walking and other locomotion on the development of bunions. Third, the present invention device is well suited for those who are prone to developing bunions, or that have a hereditary history of ancestors who have had bunions. The device can be comfortably worn for as long as the individual likes. Fourth, the present invention device helps to achieve proper toe flexion during gait. As indicated herein, it is important to tense the toe during gait to prevent the build-up of bone at the MTP joint. Fifth, the present invention device may be configured to encourage exaggerated toe-off. In extreme cases, the individual may need to concentrate and emphasize an even greater degree of toe-off for a period of time. Sixth, the present invention device may be used to teach proper biomechanics. Since some are more prone to the development of bunions than others, the present invention device may function as a teaching or training tool to break an individual of bad or improper gait patterns and to teach them more exercise more proper and therapeutic gait. Once good gait patterns are developed and these become instinctive, the device can be done away with.

The above-recited advantages are not meant to be limiting in any way. Indeed, one skilled in the art may recognize other advantages that are not specifically recited herein, each of which are intended to be included herein.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

More specifically, while illustrative exemplary embodiments of the invention have been described herein, the present invention is not limited to these embodiments, but includes any and all embodiments having modifications, omissions, combinations (e.g., of aspects across various embodiments), adaptations and/or alterations as would be appreciated by those in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given above.

What is claimed and desired to be secured by Letters Patent is:

1. A device configured to promote toe-off during gait comprising:
a first sensor located proximate the ball of a foot of an individual and configured to sense a force acting on said ball of said foot during a gait cycle and to provide a signal corresponding to said force;
a second sensor located proximate a pressure receiving surface of a toe of said foot of said individual and configured to measure a force acting on said toe of said foot during said gait cycle and to provide a signal corresponding to said force;
a control center configured to process said signals, as received from said first and second sensors, to obtain respective first and second measured values, said control center being adapted to compare said first and second measured values to at least one pre-determined value stored within said control center; and
a feedback mechanism operably connected to and controlled by said control center adapted to notify said individual of improper toe-off during said gait cycle, said feedback mechanism being activated depending upon the results of a comparison of said measured values with said at least one pre-determined value, said feedback mechanism making said individual aware of a need to correct a future gait cycle.

2. The device of claim 1, further comprising an interface medium configured to interface with said foot of said individual and to appropriately support at least said first and second sensors, and optionally said control center and said feedback mechanism.

3. The device of claim 2, wherein said interface medium is adapted to support said control center and said feedback mechanisms.

4. The device of claim 2, wherein said interface medium comprises an insert configured for placement within a shoe.

5. The device of claim 1, further comprising a power source configured to power said control center, said sensors, and said feedback mechanism.

6. The device of claim 2, further comprising means for releasably coupling said interface medium to a part of said foot.

7. The device of claim 6, wherein said means for coupling comprises an adhesive configured to facilitate repeated application and removal of said interface medium to said foot.

8. The device of claim 6, wherein said means for coupling comprises an elastic band element secured to said interface medium and operable to couple to one or more toes.

9. The device of claim 1, wherein said first and second measured values correspond to first and second signals as output from said first and second sensors, respectively, which first and second signals correspond to respective peak pressure magnitudes as registered during a current gait cycle.

10. The device of claim 1, wherein said first and second signals used to obtain said first and second measured values each correspond to respective peak pressure magnitudes as registered during a current gait cycle.

11. The device of claim 1, wherein said first and second measured values correspond to an average of a plurality of signals as output from said first and second sensors, respectively, registered during a current gait cycle.

12. The device of claim 1, wherein said feedback mechanism is activated based on a comparison falling within an unacceptable range of an average number of measured values and said pre-determined value, said average number of measured values being registered over a pre-determined number of gait cycles.

13. The device of claim 12, wherein said average number of measured values is obtained by averaging respective peak pressure magnitudes.

14. The device of claim 12, wherein said average number of measured values is obtained by averaging, respectively, a plurality of pressure magnitudes.

15. The device of claim 1, wherein said control center is configured to activate said feedback mechanism based on a differential of measured values of said first and second sensors registered during a current gait cycle as compared to said pre-determined value.

16. The device of claim 1, wherein said interface medium comprises an elastomeric material makeup that flexibly supports at least one of said sensors, said control center, said feedback mechanism, and each of their interconnects.

17. The device of claim 1, wherein said control center is configured to process said first and second measured values to obtain a measured pressure differential existing between said first and second measured values, said control center further processes said measured pressure differential for comparing to a range of pre-determined pressure differential values as stored in said control center for the purpose of determining whether to activate said feedback mechanism.

18. The device of claim 1, wherein said control center is configured to process said first and second measured values and to compare only said second value to a pre-determined value for the purpose of determining whether to activate said feedback mechanism.

19. The device of claim 1, wherein said control center is configured to process said first and second measured values and to compare these two values against each other to determine if they fall within acceptable or unacceptable ranges as based on the reading of the other.

20. The device of claim 1, wherein said control center comprises a plurality of acceptable and unacceptable values and ranges of values to be used for comparison purposes.

21. A device configured to promote toe-off during gait comprising:
a first sensor located proximate the ball of a foot of an individual and configured to sense the force acting on said ball of said foot during a gait cycle and to provide a signal corresponding to said force;
a second sensor located proximate a pressure receiving surface of a toe of said foot of said individual and configured to measure the force acting on said toe of said foot during said gait cycle and to provide a signal corresponding to said force;
a control center electrically connected to said first and second sensors, and configured to process said signals, as received from said first and second sensors, to obtain respective first and second measured values, said control center configured to compare said first and second measured values and to activate a feedback device depending upon the results of said comparison; and
an interface medium configured to house and support said first and second sensors, and optionally said control center.

22. A method for promoting toe-off of an individual during gait comprising:
positioning a first sensor proximate a ball of a foot of said individual;
sensing a force acting on said ball of said foot during a gait cycle;
providing a signal corresponding to said force;
positioning a second sensor proximate a toe of said foot;
sensing a force acting on said toe during said gait cycle;
providing a signal corresponding to said force;
transmitting said signals to a control center for processing;
assigning measured values to said signals;
comparing said measured values to one or more pre-determined values; and
activating a feedback mechanism notifying said individual of improper toe-off in the event comparison of said differential value to said pre-determined value is unacceptable as pre-determined.

23. The method of claim 22, wherein one or more measured values of said first sensor are compared to one or more pre-determined values, and wherein one or more measured values from said second sensor are separately compared to separate one or more pre-determined values.

24. The method of claim 23, wherein said feedback mechanism is triggered in the event said two separately compared measured values fall within acceptable or unacceptable ranges as based on the reading of the other.

25. The method of claim 22, wherein said measured values from said first and second sensors are compared to each other to determine a measured differential that is then compared to one or more pre-determined values.

26. The method of claim 22, wherein said feedback mechanism is activated in the event a measured value from said second sensor is below a certain threshold value.

27. A method for promoting toe-off of an individual during gait
sensing a first force applied to a ball of a foot during a gait cycle on an individual;
sensing a second force applied to a toe of said foot during said gait cycle;
identifying a differential value between said first and second signals; and
comparing said differential value with a reference value to identify improper toe-off; and
notifying said individual in the event comparison of said differential value and said reference value is unacceptable.

28. The method of claim 27, wherein said step of comparing may be customized to accommodate different users.
